(19) United States
(12) Patent Application Publication
(10) Pub. No.: US 2022/0031737 A1
(43) Pub. Date: Feb. 3, 2022

(54) PHARMACEUTICAL COMPOSITIONS COMPRISING CHROMIUM AND CARBOHYDRATE BLOCKERS

(71) Applicant: GluCare LLC, Grand Prairie, TX (US)

(72) Inventors: Harry G. Preuss, Fairfax Station, VA (US); Richard Scheckenbach, Camas, WA (US)

(73) Assignee: GluCare LLC, Grand Prairie, TX (US)

(21) Appl. No.: 17/500,332

(22) Filed: Oct. 13, 2021

Related U.S. Application Data
(62) Division of application No. 16/324,071, filed on Feb. 7, 2019.
(60) Provisional application No. 62/371,937, filed on Aug. 8, 2016.

(51) Int. Cl. A61K 33/24; A61K 31/7004; A61K 36/185; A61K 36/42; A61K 36/48; A61K 36/752; A61K 31/455; A61K 45/06; A61P 3/08

(52) U.S. Cl. CPC A61K 33/24; A61K 31/7004; A61K 36/185; A61K 36/42; A61P 3/08; A61K 36/752; A61K 31/455; A61K 45/06; A61K 36/48

(57) ABSTRACT

The present disclosure relates to pharmaceutical compositions comprising chromium, a carbohydrate blocker, and a pharmaceutically acceptable excipient; methods for supporting healthy blood sugar levels comprising administering, to a subject in need thereof, a pharmaceutical composition comprising chromium, a carbohydrate blocker, and a pharmaceutically acceptable excipient; and food or beverage additives comprising chromium and a carbohydrate blocker.

The present application claims the benefit of U.S. Provisional Application No. 62/371,937, filed on Aug. 8, 2016, which is incorporated herein by reference in its entirety.

The present disclosure relates to a pharmaceutical composition comprising chromium, a carbohydrate blocker, and a pharmaceutically acceptable excipient.

The disclosure also relates methods for supporting healthy blood sugar levels comprising administering, to a subject in need thereof, a pharmaceutical composition comprising chromium, a carbohydrate blocker, and a pharmaceutically acceptable excipient.

Insulin is a hormone produced in the beta islet cells of the pancreas. Insulin plays a significant role in metabolism by aiding glucose uptake in cells. See, e.g., Saltiel, A. R. and Kahn, C. R. Nature 414(6865): 799-806 (2001). After carbohydrates such as sugars and starches are consumed, the human digestive tract breaks down the carbohydrates primarily into glucose. When blood glucose levels rise after consuming a meal, the pancreas releases insulin into the bloodstream. Insulin and glucose are then transported to cells throughout the body, with insulin facilitating cellular intake of glucose and lowering blood glucose levels. In addition, insulin stimulates the liver and muscle tissue to store excess blood glucose. In a healthy individual with normal insulin sensitivity, these functions allow both blood glucose and insulin levels to remain in the normal range.

In a person with insulin resistance however, cells do not respond normally to insulin, and cellular uptake of glucose is less efficient. See, e.g., Kahn. B. B. and Flier, J. S. JCI 106(4) 473-481 (2000). Because cells do not absorb glucose as efficiently under these conditions, the beta cells in the pancreas usually produce increasing amounts of insulin in an attempt to lower blood glucose levels. In some cases, the pancreas is able to produce enough insulin to overcome the effects of insulin resistance, facilitating sufficient cellular uptake of glucose and maintenance of normal glucose levels.

Over time, however, beta cells in the pancreas fail to keep up with the body's increased need for insulin, and excess glucose accumulates in the bloodstream, leading to higher circulating glucose levels, prediabetes, and other serious health disorders, including accelerated aging. The U.S. Department of Health and Human Services estimated that at least 86 million U.S. adults ages 20 or older in 2012 had prediabetes, a condition in which blood glucose levels are higher than normal but not high enough for a diagnosis of diabetes, and that estimate has risen annually. See the Center for Disease Control's National Diabetes Statistics Report, 2014. In addition to diabetes and prediabetes, insulin resistant individuals are at increased risk of developing dyslipidemia, hypertension, atherosclerosis, endothelial dysfunction, microalbuminuria, obesity, depression, metabolic syndrome, and polycystic ovary syndrome.

Furthermore, glucose-insulin perturbations, particularly insulin resistance, contribute to the increasing prevalence of metabolic syndrome. In non-diabetic individuals, fasting glucose levels correlate significantly in an unhealthful direction with many components of metabolic syndrome. See, e.g., Smyth et al. Nat Med 12:75-80 (2005) and Bremer et al. Pediatrics 129:557-570 (2012). In fact, with fasting glucose in the non-diabetic range as the independent variable, the following correlations with components of metabolic syndrome are statistically significantly positive: body weight, body fat mass, systolic/diastolic BP, HbA1C, WBC/neutrophil count, and circulating levels of insulin, triglycerides, hsCRP, ALT, and globulins. See Preuss et al. Original Internist: 78.

Diet and lifestyle choices play a significant role in maintaining healthy blood glucose levels, and, consequently, reducing the risk of developing many health conditions such as type 2 diabetes and metabolic syndrome. Rapid gastrointestinal absorption of refined carbohydrates has been linked to perturbed glucose-insulin metabolism, including insulin resistance. Yudkin et al. were among the first to promote the concept that table sugar (sucrose) is a major contributor to the obesity epidemic. See Yudkin, J. Lancet 2:794 (1983). In addition, Yudkin et al. demonstrated that perturbations in insulin metabolism are associated with elements of the metabolic syndrome. Id.

Additional work has validated the role of insulin system perturbations such as insulin resistance in chronic conditions, including obesity. The role of the insulin system in determining whether fat or muscle predominates in a weight loss regimen was demonstrated in a study examining the effects of supplementation with oral niacin-bound chromium (NBC), which has been demonstrated to ameliorate insulin resistance in some patients. In a double blind, placebo-controlled, crossover study on overweight African-American females, a regimen of caloric restriction and exercise combined with NBC supplementation increased fat loss and reduced muscle loss significantly compared to a control group that did not receive the NBC supplement. See Crawford, V. et al. Diabetes, Obesity, and Metabolism 1:331-337 (1999).

Trivalent chromium, of which NBC is one form, is a nutritionally essential trace element that may be useful in the prevention of chronic conditions associated with insulin resistance. Chromium is essential for optimal insulin activity in all known insulin-dependent systems, and chromium supplementation has often led to improved blood glucose, insulin, and lipids in people with impaired glucose tolerance and/or diabetes. See, e.g., Boyle et al., Southern Med. J., 70:1449-1453, (1977). Furthermore, supplementation of chromium in people with normal blood glucose levels has been reported to lead to improvements in glucose tolerance, serum lipid concentrations, including high-density lipoprotein cholesterol, insulin and insulin binding. However, the typical Western diet does not include a significant amount of chromium, and most Americans consume far less than the upper limit of the estimated safe and adequate daily dietary intake (ESADDI), which is set at 50 to 200 μg of chromium per day. See, e.g., Anderson R. A. Reg Toxicol Pharmacol 26:S35-S41 (1997). Insufficient dietary chromium consumption can result in biologically ineffective insulin and compromised glucose metabolism, and chromium deficiency has been linked to both maturity-onset diabetes and to cardiovascular diseases. Moreover, the ESADDI may underestimate the amount of chromium that may be safely consumed to aid in the maintenance of healthy blood sugar levels. For example, in one Chinese study, administration of 1,000 μg of chromium per day (five times above the upper limit of the ESADDI) was shown to be highly effective for relieving many symptoms of type 2 diabetes.

Chromium is widely considered to be an insulin sensitizer. See, e.g., Preuss, H. G and Anderson, R. A. Current Opinion in Clinical Nutrition & Metabolic Care 1(6):509-512 (1998). Insulin sensitizers help the body lower blood sugar by increasing the muscle, fat, and liver's ability to take up glucose. While the exact mechanism of action for chromium is not known, it has been shown to have an effect on insulin receptor number. Chromium functions as a co-factor for the action of insulin, binding to insulin and potentiating some, and possibly all, insulin functions. However, while chromium has been shown to decrease the symptomatic manifestation of metabolic syndrome (e.g., the portion of systolic blood pressure elevated by high sucrose intake), high levels of sucrose ingestion eventually overcome the healthy effects of chromium supplementation. By lowering carbohydrate load, it may he possible to increase and prolong the healthy effects of chromium supplementation in individuals with insulin resistance or at risk of developing insulin resistance.

While adherence to a diet low in refined carbohydrates (“low carb diet”), especially rapidly absorbed refined carbohydrates with a high glycemic index, is a straightforward method for lowering carbohydrate load, many individuals are not prepared to accept this lifestyle change. Issues ranging from the wisdom of replacing dietary carbohydrates with fat to the palatability of low carb diets and possible addiction to refined carbohydrates have limited widespread adoption of low carb diets. Increased ingestion of viscous fibers is an alternative method for slowing down the absorption of carbohydrates and maintaining better insulin sensitivity. However, adoption of fiber rich diets has also been limited, possibly due to the gastrointestinal disturbances that may be associated with fiber rich diets in many people.

The use of dietary supplements comprising inhibitors of carbohydrate absorption (“carbohydrate blockers”) may be a more palatable alternative to a low carb and/or fiber rich diets for many individuals. See, e.g., Preuss, H. G. Journal of the American College of Nutrition 28(3):266-276 (2009). Complex carbohydrates (starches) cannot be absorbed unless they are first broken down by the digestive enzyme amylase. When amylase is blocked, complex carbohydrates may pass through the body undigested. Many natural carbohydrate blockers have been discovered that inhibit absorption of carbohydrates by inhibiting enzymes associated with absorption such as α-amylase and α-glucosidase, as well by at least partially inhibiting glucose or fructose transportation in the gastrointestinal tract. These natural enzyme inhibitors (amylase/sucrase) reportedly lessen breakdown of starches and sucrose, limiting gastrointestinal absorption. For example, it has been known for decades that certain bean extracts such as white kidney bean extract can at least partially inhibit the enzyme α-amylase, slowing down the absorption of both starch and sucrose. In previous rat studies, Hibiscus extract, another natural carbohydrate blocker that inhibits α-amylase, proved to be as safe and effective as white kidney bean extract. See Preuss, H. G. et al. Int J Med Sci 4(4):196-202 (2007). In addition, L-arabinose, a non-calorie natural compound sweetener with similarities to glucose, is known to function as a sucrase inhibitor that slows sucrose absorption. See Seri, K., et al. Metabolism 45(10:1368-1374 (1996). By reducing the rate of glucose metabolism, L-arabinose may result in a more sustained release of energy and natural regulation of blood glucose (i.e., avoidance of insulin spikes).

The effects of white kidney bean extract, Hibiscus extract, Momordica charantia extract, and L-arabinose on glucose levels in rats have been studied individually. For example, in one study, Sprague-Dawley rats were gavaged with water or water plus rice starch and/or sucrose, and circulating glucose was measured at timed intervals thereafter. Glucose elevations above baseline over four hours following rice starch challenge were 40%, 27%, and 85% of control after ingesting bean extract, Hibiscus extract, and L-arabinose, respectively. See Preuss, H. G. et al. Int J Med Sci 4(4):196-202 (2007).

Additionally, Momordica charantia extract, also known as bitter melon, bitter apple, bitter gourd, or bitter cucumber, may have glucose-lowering properties.

Furthermore, Hibiscus extract, a potent source of polyphenols, has been effectively used to treat high blood pressure, diabetes, and liver disorders.

In addition, the effects of white kidney bean extract on human carbohydrate absorption have been investigated. For example, Vinson et al. previously carried out single dose human studies. In one study, eleven fasting subjects were given sliced white bread and 42 grams of margarine with and without 1.5 grams of bean extract. See Vinson, J. A. et al. Open Nutraceutical Journal 2:88-91 (2009). Absorption of carbohydrates was decreased by 66% via area under the curve estimation for the group given bean extract relative to the control group. In a separate study, 39 obese subjects (BMI 30-43) were randomly allocated to receive either 1500 mg of bean extract or placebo. See Udani, J. et al. Altern Med Rev 9:63-69 (2004). Twenty-seven subjects completed the study (14 active and 13 placebo). The subjects were instructed to consume the test product with lunch and dinner each day for eight weeks. The test product was administered with at least 8 oz. of water. Subjects began a controlled high fiber/low fat diet at the beginning of the study that provided 100 g to 200 g of complex carbohydrates daily. Subjects were instructed to eat the majority of these carbohydrates during lunch and dinner. Based on an intent-to-treat analysis, the treatment group lost an average of 3.79 lbs. (an average of 0.47 lbs. per week) compared with the placebo group that lost an average of 1.65 lbs. (an average of 0.21 lbs. per week) (p=0.35) during the eight week study. In addition, triglyceride levels in the treatment group were reduced by an average of 26.3 mg/dL compared to the 8.2 mg/dL decrease in the placebo group (p=0.07).

By administering chromium, an insulin sensitizer, with carbohydrate blockers, especially combinations of carbohydrate blockers that inhibit carbohydrate absorption via different mechanisms, the effects of chromium may be increased by reducing the carbohydrate load. In addition, by reducing the carbohydrate load, pharmaceutical compositions comprising chromium, a carbohydrate blocker, and a pharmaceutically acceptable excipient may support insulin function and activity and assist in the maintenance of healthy levels of blood glucose. While it is anticipated that the greatest improvements will be experienced by subjects who add pharmaceutical compositions comprising chromium, a carbohydrate blocker, and a pharmaceutically acceptable excipient to a lifestyle comprising a healthy diet and exercise, individuals who are unwilling or unable to make lifestyle modifications may also experience improved health outcomes.

For example, the most common experimental means to lengthen life span, caloric restriction, appears to work, at least in part, through its beneficial effects on the insulin system. See Masoro E. J. et al, J. Gerontology 47:B202-B208 (1992). Aging is associated with elevated blood glucose and circulating insulin, insulin resistance, elevated cholesterol and triglycerides, decreased nerve conduction, and decreased lean body mass. Interestingly, all these perturbations are also associated with chromium deficiency. See Anderson R. A., J Amer Coll Nutr 16:404-410 (1997). By supporting insulin function and activity, as well as aiding in the maintenance of healthy levels of blood glucose, pharmaceutical compositions comprising chromium, a carbohydrate blocker, and a pharmaceutically acceptable excipient may lead to an increased life span, reduced insulin resistance, and a reduced incidence of chronic conditions associated with metabolic syndrome.

Moreover, administration of a pharmaceutical composition comprising chromium and a carbohydrate blocker may increase high-density lipoproteins (HDL) levels in patients in need thereof.

The present disclosure relates to a pharmaceutical composition comprising chromium, a carbohydrate blocker, and a pharmaceutically acceptable excipient. In some embodiments, the pharmaceutical composition further comprises an energy-boosting additive, which may, for example, lead to increased compliance with a dosing regimen. Moreover, energy-boosting additives such as Seville orange extract may also enable the body's metabolism to more rapidly burn fat for energy. Seville orange extract, a combination of several alkaloids including synephrine, N-methyltyramine, hordenine, octopamine, and tyramine, may target specific receptors in the body that promote metabolism. These pharmaceutical compositions have active ingredients useful for at least partially blocking digestion and adsorption of dietary carbohydrates.

The present disclosure also relates to a food or beverage additive comprising chromium and a carbohydrate blocker. Some embodiments of the disclosure are directed to food or beverage additives comprising chromium, L-arabinose, Hibiscus extract, and white kidney bean extract. Some embodiments further comprise Momordica charantia extract and/or Seville orange extract.

The present disclosure also relates to methods of use of said pharmaceutical compositions, including the treatment of several diseases, disorders, or conditions as described herein. Some embodiments of the disclosure relate to methods for supporting healthy blood sugar levels comprising administering a pharmaceutical composition comprising chromium, a carbohydrate blocker, and a pharmaceutically acceptable excipient. In some embodiments of said methods, the pharmaceutical composition comprises chromium, L-arabinose, white kidney bean extract, Hibiscus extract, and a pharmaceutically acceptable excipient, In some embodiments of said methods, the pharmaceutical composition comprises chromium, L-arabinose, white kidney bean extract, Hibiscus extract, Momordica charantia extract, and a pharmaceutically acceptable excipient. In some embodiments, pharmaceutical compositions described herein further comprise Seville orange extract.

Some embodiments of the disclosure also relate to methods for treating, reducing the severity of, reducing the incidence of, delaying the onset of, or reducing pathogenesis of a chronic condition associated with metabolic syndrome comprising administering, to a subject in need thereof, a pharmaceutical composition comprising chromium, a carbohydrate blocker, and a pharmaceutically acceptable excipient. In some embodiments of said methods, the pharmaceutical composition comprises chromium, L-arabinose, Hibiscus extract, white kidney bean extract, and a pharmaceutically acceptable excipient.

Some embodiments of the disclosure relate to methods for treating, reducing the severity of, reducing the incidence of, delaying the onset of, or reducing pathogenesis of insulin resistance comprising administering, to a subject in need thereof, a pharmaceutical composition comprising chromium, a carbohydrate blocker, and a pharmaceutically acceptable excipient. In some embodiments of said methods, the pharmaceutical composition comprises chromium, L-arabinose, Hibiscus extract, white kidney bean extract, and a pharmaceutically acceptable excipient.

### DETAILED DESCRIPTION

Unless otherwise defined, all technical and scientific terms used herein possess the meaning commonly understood by the skilled artisan. In the case of inconsistencies, the present disclosure, including definitions, controls.

As used above, and throughout the description, the following terms, unless otherwise indicated, shall be understood to have the following meanings.

As used here, “a” (or “an”), “one or more,” and “at least one” can be used interchangeably and refer to one or more an entity. For example, a carbohydrate blocker refers to one or more carbohydrate blockers or at least one carbohydrate blocker.

As used herein, “about” means within 10%, such as within 5% and further such as within 2.5%, of a given value or range. When the term “about” is used in conjunction with a numerical range, it modifies that range by extending the boundaries above and below the numerical values set forth.

As used herein, an “active ingredient” is an ingredient in a pharmaceutical composition that is biologically active (i.e., alters a chemical or physiological function of a cell, tissue, organ, or organism).

As used herein, a “pharmaceutically acceptable excipient” is a functional or non-functional ingredient in a pharmaceutical composition other than the active ingredient(s) useful in preparing said pharmaceutical composition. A “pharmaceutically acceptable excipient” is generally safe and acceptable for mammalian pharmaceutical use.

As used herein, a “disintegrant” is a pharmaceutically acceptable excipient that hydrates a pharmaceutical composition and facilitates the disintegration or breakup of a pharmaceutical composition (e.g., a tablet).

As used herein, a “diluent” or “filler” is an excipient that dilutes the active ingredient(s) and adds bulkiness to a pharmaceutical composition. For example, a diluent or filler may stabilize the active ingredient(s) or facilitate compression.

As used herein, a “surfactant” is an excipient that imparts pharmaceutical compositions with enhanced solubility and/or wettability.

As used herein, a “hinder” is a pharmaceutically acceptable excipient that imparts a pharmaceutical composition with cohesive qualities or tensile strength (e.g., hardness).

As used herein, a “glidant” is a pharmaceutically acceptable excipient that imparts a pharmaceutical composition with enhanced flow properties, thereby preventing, reducing, or inhibiting adhesion or friction during processing.

As used herein, a “lubricant” is a pharmaceutically acceptable excipient that imparts improved compaction and ejection properties to a pharmaceutical composition by preventing the active ingredients) from clumping together and sticking to manufacturing equipment.

As used herein, “encapsulation machinery” refers to any machine or piece of equipment that may be used to facilitate capsule filling. Encapsulation machinery may be automatic, semiautomatic, or manual.

As used herein, “tableting machinery” refers to any machine or piece of equipment that may be used to facilitate tablet production. Tableting machinery may he automatic, semiautomatic, or manual.

As used herein, a “carbohydrate blocker” is a compound that slows the gastrointestinal absorption of a carbohydrate. Non-limiting examples of carbohydrate blockers include α-amylase inhibitors, α-glucosidase inhibitors, glucose transport inhibitors, and fructose transport inhibitors.

As used herein, an “α-amylase inhibitor” is a compound that at least partially inhibits the activity of an α-amylase enzyme (e.g., via competitive inhibition, noncompetitive inhibition, uncompetitive inhibition, mixed inhibition, or irreversible covalent inhibition).

As used herein, an “α-glucosidase inhibitor” is a compound that at least partially inhibits the activity of an α-glucosidase enzyme (e.g., via competitive inhibition, noncompetitive inhibition, uncompetitive inhibition, mixed inhibition, or irreversible covalent inhibition).

As used herein, a “glucose transport inhibitor” is a compound that inhibits the transport of glucose by at least partially inhibiting the activity of at least one glucose transporter (e.g., via competitive inhibition, noncompetitive inhibition, uncompetitive inhibition, mixed inhibition, or irreversible covalent inhibition).

As used herein, a “fructose transport inhibitor” is a compound that inhibits the transport of fructose by at least partially inhibiting the activity of at least one fructose transporter via competitive inhibition, noncompetitive inhibition, uncompetitive inhibition, mixed inhibition, or irreversible covalent inhibition.

As used herein, “% w/w” refers to the weight percentage of an ingredient in a pharmaceutical composition. For example, 5% w/w means that the weight of an ingredient is 5% of the total weight of the pharmaceutical composition. The total weight of the pharmaceutical composition includes the weight of the ingredient.

As used herein, an “extract” of an entity is a substance produced by subjecting the entity to an extraction process, typically using a extraction solvent such as ethanol. As a non-limiting example, white kidney bean extract may be prepared by grounding white kidney beans and extracting with water as an extraction solvent. See, e.g., Barrett and Udani, Nutr J. 10(1): 1 (2011).

As used herein, a “food or beverage additive” is a compound or composition that may be added to a food or beverage product. As a non-limiting example, a food or beverage additive may be added to the surface of a food product. In another non-limiting example, a food or beverage additive may be homogeneously or heterogeneously dispersed in a food or beverage product.

As used herein, “daily dosage” refers to the total quantity of an active ingredient consumed in the form of a pharmaceutical composition. As used herein, the daily dosage of an active ingredient does not include active ingredient consumed via normal eating behaviors (i.e., dietary sources of the active ingredient).

As used herein, “metabolic syndrome” refers to a cluster of health conditions including increased blood pressure, high blood sugar, excess body fat around the waist, and abnormal cholesterol or triglyceride levels. According to guidelines used by the National Institute of Health, a subject has metabolic syndrome if the subject has three or more of the following traits or takes medication to control them:
Large waist circumference—a waistline that measures at least 35 inches (89 centimeters) for women and 40 inches (102 centimeters) for men
High triglyceride level—150 milligrams per deciliter (mg/dL), or 1.7 millimoles per liter (mmol/L), or higher of this type of fat found in blood
Reduced high-density lipoprotein (HDL) cholesterol—less than 40 mg/dL (1.04 mmol/L) in men or less than 50 mg/dL (1.3 mmol/L) in women
Increased blood pressure—130/85 millimeters of mercury (mm Hg) or higher
Elevated fasting blood sugar—100 mg/dL (5.6 mmol/L) or higher

As used herein, a “chronic condition associated with metabolic syndrome” refers to persistent or otherwise long-lasting health conditions (e.g., lasting for more than three months) frequently found in subjects with metabolic syndrome. Non-limiting examples of chronic conditions associated with metabolic syndrome include insulin resistance, type 2 diabetes, obesity, nonalcoholic fatty liver disease, chronic kidney disease, elevated blood pressure, polycystic ovary syndrome, cardiovascular disorders such as hypertension, and dyslipidemias such as high triglyceride and low HDL-cholesterol levels. Other non-limiting examples of chronic conditions associated with metabolic syndrome include acanthosis nigricans, hirsutism, peripheral neuropathy, and retinopathy.

As used herein, “diabetic fasting glucose levels” refer to blood glucose levels of about 126 mg/dL (7 mmol/L) or higher following at least 8 hours of fasting in which a subject does not eat or drink anything except for water.

As used herein, “non-diabetic fasting glucose levels” refer to blood glucose levels less than about 126 mg/dL (7 mmol/L) following at least 8 hours of fasting in which a subject does not eat or drink anything except for water.

As used herein, “prediabetic fasting glucose levels” refer to blood glucose levels between about 100 mg/dL (5.6 mmol/L) and about 125 mg/dL (6.9 mmol/L) following at least 8 hours of fasting in which a subject does not eat or drink anything except for water.

In one aspect, the present disclosure provides a pharmaceutical composition comprising chromium, a carbohydrate blocker, and a pharmaceutically acceptable excipient.

In some embodiments, the chromium is chromium nicotinate, chromium polynicotinate, chromium picolinate, chromium histidinate, or a mixture thereof. For example, in some embodiments, the chromium is chromium polynicotinate. In some other embodiments, the chromium is a 1:1 mixture of chromium picolinate and chromium histidinate.

In some embodiments, the carbohydrate blocker is an α-amylase inhibitor, an α-glucosidase inhibitor, a glucose transport inhibitor, a fructose transport inhibitor, or a mixture thereof. Non-limiting examples of an α-amylase inhibitor include Syzygium cumini extract, Psidium guajava extract, Amaranthus caudatus extract, Balanites aegyptiaca extract, Camellia sinensis extract, Galega officinalis extract, Holarrhena floribunda extract, Khaya senegalensis extract, Melissa officinalis extract, Mitragyna inermis extract, Rosmarinus officinalis extract, Securidaca longepedunculata extract., Tamarindus indica extract, Taraxacum officinale extract, Vaccinium myrtillus extract, Rhodiola rosea extract, Ribes pullchelum extract, Vaccinium uliginosum extract, Geranium pretense extract, Leontopodium ochroleucum extract, Paeonia anomala extract, Pentaphylloides fruticosa extract, acarbose, fisetin, luteolin, rosmarinic acid, daidzein, and bean extract such as white kidney bean extract, In some embodiments, the carbohydrate blocker is an α-amylase inhibitor and an α-glucosidase inhibitor.

Non-limiting examples of an α-glucosidase inhibitor include acarbose, miglitol, L-arabinose, Gymnema sylvestre extract, Momordica charantia extract, Trrigonella foenum gracecum extract, Pterocarpus marsupium extract, Murraya koenigii extract, Ocimum sanctum extract, Tinospora cordifolia extract, Szygium cumini extract, Zingiber officinale extract, and Allium sativum extract.

Non-limiting examples of a glucose transport inhibitor include apple extract, phloridzin, Gymnema sylvestre extract, strawberry extract, and green coffee bean extract.

Non-limiting examples of a fructose transport inhibitor include eucalyptus extract and coffee extract.

In some embodiments, the carbohydrate blocker is selected from white kidney bean extract, L-arabinose, Hibiscus extract, Momordica charantia extract, Gymnema extract, apple extract, and eucalyptus extract.

In some embodiments, the carbohydrate blocker is white kidney bean extract. White kidney bean extract is also referred to as the extract of cannellini bean, bush bean, and common bean. In some embodiments, white kidney bean extract is extracted from Phaseolus vulgaris fruit. In some embodiments, white kidney bean extract is a water extract of Phaseolus vulgaris.

In some embodiments, the carbohydrate blocker is L-arabinose.

In some embodiments, the carbohydrate blocker is Hibiscus extract. Hibiscus extract is also referred to as the extract of roselle, red Sorrel, Jamaican sorrel, or Flor de Jamaica. In some embodiments, Hibiscus extract is extracted from Hibiscus sabdariffa fruit. In some embodiments, Hibiscus extract is a water extract from Hibiscus sabdariffa.

In some embodiments, the carbohydrate blocker is Momordica charantia extract. In embodiments of the present disclosure, Momordica charantia extract may function as an α-glucosidase/α-amylase inhibitor, a sugar-blocker (e.g., by inhibiting gastrointestinal absorption of glucose), or as an insulin sensitizer.

In some embodiments, the carbohydrate blocker is white kidney bean extract and L-arabinose. In some embodiments, the carbohydrate blocker is Hibiscus extract and L-arabinose. In some embodiments, the carbohydrate blocker is white kidney bean extract, L-arabinose, and Hibiscus extract.

In some embodiments, the carbohydrate blocker is Momordica charantia extract and white kidney bean extract. In some embodiments, the carbohydrate blocker is Momordica charantia extract and L-arabinose. In some embodiments, the carbohydrate blocker is Momordica charantia extract and Hibiscus extract.

In some embodiments, the carbohydrate blocker is Momordica charantia extract, white kidney bean extract, and L-arabinose. In some embodiments, the carbohydrate blocker is Momordica charantia extract, white kidney bean extract, and Hibiscus extract. In some embodiments, the carbohydrate blocker is Momordica charantia extract, L-arabinose, and Hibiscus extract. In some embodiments, the carbohydrate blocker is Momordica charantia extract, L-arabinose, white kidney bean extract, and Hibiscus extract.

In some embodiments, chromium is present in an amount ranging from about 50 meg to about 300 mcg. For example, in some embodiments, chromium is present in amount of about 50 mcg, about 60 meg, about 70 mcg, about 80 mcg, about 90 mcg, about 100 mcg, about 110 mcg, about 120 mcg, about 130 mcg, about 140 mcg, about 150 meg, about 160 mcg, about 170 meg, about 180 meg, about 190 mcg, about 200 mcg, about 210 mcg, about 220 mcg, about 230 meg, about 240 mcg, about 250 mcg, about 260 meg, about 270 mcg, about 280 mcg, about 290 mcg, or about 300 mcg. In some embodiments, chromium is present in an amount of about 250 mcg. In some embodiments, chromium is present in an amount of about 100 mcg.

In some embodiments, white kidney bean extract is present in an amount ranging from about 300 mg to about 1000 mg. In some embodiments, white kidney bean extract is present in an amount of about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. In some embodiments, white kidney bean extract is present in an amount of about 500 mg.

In some embodiments, L-arabinose is present in an amount ranging from about 250 mg to about 500 mg. In some embodiments, L-arabinose is present in amount ranging from about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg. In some embodiments, L-arabinose is present in an amount of about 500 mg.

In some embodiments, Hibiscus extract is present in an amount ranging from about 100 mg to about 500 mg. In some embodiments, Hibiscus extract is present in an amount of about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg. In some embodiments, Hibiscus extract is present in an amount of about 200 mg.

In some embodiments, Momordica charantia extract is present in an amount ranging from about 50 mg to about 2000 mg. In some embodiments, Momordica charantia extract is present in an amount of about 50 mg, about 100 mg, about 150 mg, about 200 mg, about 250 mg, about 300 mg, about 350 mg, about 400 mg, about 450 mg, about 500 mg, about 550 mg, about 600 ma, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 ma, about 1700 mg, about 1750 mg, about 1800 mg, about 1850 mg, about 1900 mg, about 1950 mg, or about 2000 mg. In some embodiments, Momordica charantia extract is present in an amount of about 200 mg.

In another aspect, the present disclosure provides a pharmaceutical composition of claim 1 comprising:

about 50 mcg to about 300 mcg chromium;

about 300 mg to about 1000 mg white kidney bean extract;

about 250 mg to about 500 mg L-arabinose;

about 100 mg to about 500 mg Hibiscus extract; and

a pharmaceutically acceptable excipient.

In some embodiments, the pharmaceutical composition comprises:

about 250 mcg chromium;

about 500 mg white kidney bean extract;

about 500 mg L-arabinose;

about 200 mg Hibiscus extract; and

a pharmaceutically acceptable excipient.

In another aspect, the present disclosure provides a pharmaceutical composition of claim 1 comprising:

about 50 mcg to about 300 mcg chromium;

about 300 mg to about 1000 mg white kidney bean extract;

about 250 mg to about 500 mg L-arabinose;

about 100 mg to about 500 mg Hibiscus extract;

about 50 mg to about 2000 mg Momordica charantia extract; and

a pharmaceutically acceptable excipient.

In another aspect, the present disclosure provides a pharmaceutical composition of claim I comprising:

about 100 mcg chromium;

about 500 mg white kidney bean extract;

about 500 mg L-arabinose;

about 200 mg Hibiscus extract;

about 200 mg Momordica charantia extract; and

a pharmaceutically acceptable excipient.

In some embodiments, a pharmaceutical composition disclosed herein further comprises an energy-boosting additive. Non-limiting examples of energy-boosting additive include caffeine, guarana, green tea extract, ginseng, coffee extract, black tea extract, Oolong tea extract, vitamin B12, coenzyme Q12, Seville orange extract, or a mixture thereof. In some embodiments, the energy-boosting additive may lead to improved compliance with a dosing regimen.

In some embodiments, the energy-boosting additive is Seville orange extract. Seville orange extract is also referred to as extract of bitter orange, green orange, sour orange, or Bergamot orange. In some embodiments, the Seville orange extract is extracted from Citrus aurantium fruit. In some embodiments, the Seville orange extract is a water extract from Citrus aurantium fruit. In some embodiments, the Seville orange extract is present in an amount ranging from about 100 mg to about 200 mg. In some embodiments, the Seville orange extract is present in an amount of about 100 mg, about 110 mg, about 120 mg, about 130 mg, about 140 mg, about 150 mg, about 160 mg, about 170 mg, about 180 mg, about 190 mg, or about 200 mg. In some embodiments, the Seville orange extract is present in an amount of about 200 mg.

A person of skill in the art will appreciate that concentration of active ingredients in an extract may vary and that modifications to the daily dosage ranges for extracts may be required to account for batch-to-batch and source-to-source variability in extract concentrations.

In some embodiments, the total weight percentage of pharmaceutical excipient(s) in a pharmaceutical composition disclosed herein is up to about 35% w/w. For example, in some embodiments, the total weight percentage of pharmaceutical excipient(s) in a pharmaceutical composition disclosed herein is up to about 35% w/w, up to about 30% w/w, up to about 25% w/w, up to about 20% w/w, up to about 15% w/w, or up to about 10% w/w.

In some embodiments, a pharmaceutical composition disclosed herein is formulated as a solid oral dosage form. Non-limiting examples of solid oral dosage forms include tablets, such as a sugar-coated tablet, a film-coated tablet, a sublingual tablet, a buccal tablet, or an orally disintegrating oral tablet, and capsules, such as a soft capsule or microcapsule.

A pharmaceutical composition of the present disclosure may be produced by compacting or compressing an admixture or composition, for example, a powder or granules, under pressure to form a stable three-dimensional shape such a tablet. A solid oral dosage form of the disclosure may possess almost any shape including concave and/or convex faces, rounded or angled corners, and a rounded to rectilinear shape. In some embodiments, the solid oral dosage form may be a rounded tablet having flat faces.

In some embodiments, the solid oral dosage form is a capsule. In some embodiments, the solid oral dosage form is a hard capsule. In some embodiments, the solid oral dosage form is a soft gel capsule. The pharmaceutical composition in any capsule compartment may be present in any suitable form, e.g., as a powder, granules, compacts, or microcapsules. The contents of the compartments, e.g., drug substances, may be introduced into the compartments by standard methods used conventionally for filling capsules. The capsule material may be selected from materials acceptable for the delivery of a pharmaceutical or food composition. Non-limiting examples of suitable capsule materials are gelatin and plant based polymers.

A solid oral dosage form of the present disclosure may be prepared by any known production method generally used in the technical field of pharmaceuticals preparation. In particular embodiments, solid oral dosage forms provided herein may be prepared using conventional methods known to those skilled in the field of pharmaceutical preparation, as described, e.g., in pertinent textbooks. See, e.g., Remington: The Science and Practice of Pharmacy, 21st Ed., Lippincott Williams & Wilkins, Baltimore, Md. (2003); Ansel et al., Pharmaceutical Dosage Forms And Drug Delivery Systems, 7th Edition, Lippincott Williams & Wilkins, (1999); The Handbook of Pharmaceutical Excipients, 4th edition, Rowe et al., Eds., American Pharmaceuticals Association (2003); Gibson, Pharmaceutical Preformulation And Formulation, CRC Press (2001). These references are hereby incorporated herein by reference to the extent they disclose suitable, conventional methods known to those skilled in the field of pharmaceutical formulation.

A pharmaceutical composition of the present disclosure comprises a pharmaceutically acceptable excipient. Non-limiting examples of a pharmaceutically acceptable excipient include binders, disintegrants, fillers, glidants, lubricants, preservatives, antifoaming agents, fillers, colorants, lubricants, and plasticizers. Pharmaceutically acceptable excipients are well-known in the art, see, e.g., Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012), which is incorporated by reference to die extent it discloses lists of pharmaceutically acceptable excipients. Using methods generally used in the technical field of pharmaceutical preparations, the skilled artisan would know how to evaluate the compatibility of excipients with the active ingredients, i.e., chromium and a carbohydrate blocker, of the pharmaceutical composition.

In some embodiments, a pharmaceutical composition of the present disclosure comprises filler, a binder, a disintegrant, a lubricant, or a glidant. In some embodiments, the filler is mannitol, sorbitol, gelatin, dibasic calcium phosphate dihydrate, dibasic calcium phosphate anhydrate, and tribasic calcium phosphate, or any mixture thereof. In some embodiments, the binder is hydroxypropyl cellulose, alginic acid, carboxymethylcellulose sodium, copovidone, methylcellulose, or any mixture thereof. In some embodiments, the disintegrant is sodium starch glycolate, croscarmellose sodium, crospovidone, or any mixture thereof. In some embodiments, the lubricant is magnesium stearate, stearic acid, palmitic acid, calcium stearate, carnauba wax, hydrogenated vegetable oils, mineral oil, polyethylene glycols, or sodium stearyl fumarate. In some embodiments, the glidant is colloidal silicon dioxide.

In some embodiments, the pharmaceutically acceptable excipient comprises a filler, a disintegrant, a lubricant, and a glidant. In some embodiments, the filler is gelatin. In some embodiments, the disintegrant is microcrystalline cellulose. In some embodiments, the lubricant is magnesium stearate. In some embodiments, the glidant is silicon dioxide.

In some embodiments, the pharmaceutically acceptable excipient comprises a preservative. Non-limiting examples of preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutically acceptable excipient comprises a disintegrant. Non-limiting examples of disintegrants include a starch, e.g., a natural starch such as corn starch or potato starch, a pregelatinized starch such as National 1551 or sodium starch glycolate such as Promogel® or Explotab®, a cellulose such as a wood product, methylcrystalline cellulose, e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, Citrus pulp, sodium lauryl sulfate, sodium lauryl sulfate in combination starch, and mixtures thereof.

In certain embodiments, the pharmaceutically acceptable excipient comprises a diluent. Non-limiting examples of diluents include lactose, gelatin, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropyl-methylcellulose, hydroxypropylmethylceliulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and mixtures thereof.

In some embodiments, a pharmaceutical composition disclosed herein is formulated as one, two, or three solid oral dosage forms. In some embodiments, a pharmaceutical composition disclosed herein is formulated as one solid oral dosage forms. In some embodiments, a pharmaceutical composition disclosed herein is formulated as two solid oral dosage forms. In some embodiments, a pharmaceutical composition disclosed herein is formulated as three solid oral dosage forms.

In some embodiments, the pharmaceutical compositions disclosed herein are formulated as a powder.

In some embodiments, the pharmaceutical composition disclosed herein may be formulated as a liquid. In some embodiments, the pharmaceutical composition disclosed herein may be formulated as a liquid suitable for intravenous administration.

In another aspect, the present disclosure provides a method for producing a capsule comprising chromium, a carbohydrate blocker, and a pharmaceutically acceptable excipient, comprising the steps of:

blending chromium, the carbohydrate blocker(s), and the pharmaceutically acceptable excipient(s) into a homogeneous powder; and

transferring the powder to encapsulating machinery, and

filling the capsule using the encapsulating machinery.

In some embodiments, the encapsulating machinery is automatic, semi-automatic, or manual. Non-limiting examples of encapsulating machinery include LIQFIL super Labo Capsule Filling Machine by Qualicaps and Capsugel Ultra 8 II.

In some embodiments, the method further comprises one or more of the following steps prior to blending chromium, the carbohydrate blocker(s), and the pharmaceutically acceptable excipient(s) into a homogeneous powder:

analyzing a physical characteristic of one or more of chromium, the carbohydrate blocker(s), or the pharmaceutically acceptable excipient(s);

hygienic analysis of one or more of chromium, the carbohydrate blocker(s), or the pharmaceutically acceptable excipient(s);

purity and potency analysis of one or more of chromium, the carbohydrate blocker(s), or the pharmaceutically acceptable excipient(s).

Non-limiting examples of methods for analyzing physical characteristics include organoleptic analyses and particle size analysis.

Non-limiting examples of methods for hygienic analysis include total plate counts for microorganisms (e.g., Escherichia coil, Staphylococcus aureus, or yeast), wherein one or more of the carbohydrate blocker(s), the pharmaceutically acceptable excipient(s) is added to a sterile plate with solid growth medium and growth of microorganisms on the plate is measured over time relative to a control plate.

Non-limiting examples of methods for purity and potency analysis include high performance liquid chromatography and atomic absorption.

In some embodiments, the ambient temperature is below about 90° F. In some embodiments, the encapsulating machinery temperature is below about 90° F. In some embodiments, the ambient temperature and the encapsulating machinery temperature is below about 90° F.

In another aspect, the present disclosure provides a method for producing a tablet comprising chromium, a carbohydrate blocker, and a pharmaceutically acceptable excipient, comprising the steps of:

blending chromium, the carbohydrate blocker(s), and the pharmaceutically acceptable excipient(s) into a homogeneous powder;

transferring the powder to tableting machinery; and

compressing the powder using the tableting machinery.

In some embodiments, the method further comprises the steps of:

transferring the compressed powder to a coating pan; and

coating the tablet.

In some embodiments, the method further comprises one or more of the following steps prior to blending chromium, the carbohydrate blocker(s), and the pharmaceutically acceptable excipient(s) into a homogeneous powder:

analyzing a physical characteristic of one or more of chromium, the carbohydrate blocker(s), or the pharmaceutically acceptable excipient(s);

hygienic analysis of one or more of chromium, the carbohydrate blocker(s), or the pharmaceutically acceptable excipient(s);

purity and potency analysis of one or more of chromium, the carbohydrate blocker(s), or the pharmaceutically acceptable excipient(s).

In another aspect, the present disclosure provides a food or beverage additive comprising chromium and a carbohydrate blocker.

In some embodiments, the food or beverage additive is formulated as a powder. In some other embodiments, the food or beverage additive is formulated as a powder for distribution on the surface of a food product during manufacturing or immediately prior to food consumption. In some other embodiments, the food or beverage additive is formulated as a powder for dissolution in a beverage. In some other embodiments, the food or beverage additive is formulated as a powder for use in cooking or baking.

In some embodiments, the food or beverage additive is formulated as a liquid. In some other embodiments, the food or beverage additive is formulated as a liquid for use in cooking or baking. In some embodiments, the food or beverage additive is formulated as a liquid for addition to beverages.

In some embodiments, the food or beverage additive is intended for use in a beverage comprising carbohydrates. For example, in some embodiments, the beverage comprising carbohydrates is selected from soda, milk, alcoholic beverages such as beer and wine, and fruit juice.

In some embodiments, the food or beverage additive is intended for use in cooking or baking a food comprising carbohydrates. For example, in some embodiments, the food comprising carbohydrates is selected from bread, rice, pasta, potato chips, pastries, cake, cookies, oatmeal, beans, potatoes, pies, popcorn, and doughnuts.

In some embodiments, the food or beverage additive is intended for distribution on the surface of a food product. For example, in some embodiments, the food or beverage additive is intended for distribution on the surface of fruit, vegetables, bread, rice, pasta, potato chips, pastries, cake, cookies, oatmeal, beans, potatoes, pies, popcorn, ice cream, and doughnuts.

In some embodiments, the present disclosure provides a food or beverage additive comprising chromium and a carbohydrate blocker, wherein the chromium is chromium nicotinate, chromium polynicotinate., chromium picolinate, chromium histidinate, or a mixture thereof. In some embodiments, the chromium is chromium polynicotinate.

In some embodiments, the present disclosure provides a food or beverage additive comprising chromium and a carbohydrate blocker, wherein the carbohydrate blocker is an α-amylase inhibitor, an α-glucosidase inhibitor, a glucose transport inhibitor, a fructose transport inhibitor, or a mixture thereof.

In some embodiments, the α-amylase inhibitor is Syzygium cumini extract, Psidium guajava extract, Amaranthus caudatus extract, Balanites aegyptiaca extract, Camellia sinensis extract, Galega officinalis extract, Holarrhena floribunda extract, Khaya senegalensis extract, Melissa officinalis extract, Mitragyna inermis extract, Rosmarinus officinalis extract, Securidaca longepedunculata extract., Tamarindus indica extract, Taraxacum officinale extract, Vaccinium myrtillus extract, Rhocliola rosea extract, Ribes pullchelum extract, Vacciniuni uliginosum extract, Geranium pretense extract, Leontopodium ochroleucum extract, Paeonia anomala extract, Pentaphylloides fruticosa extract, acarbose, fisetin, luteolin, rosmarinic acid, daidzein, bean extract such as white kidney bean extract, or a mixture thereof. In some embodiments, the α-amylase inhibitor is white kidney bean extract.

In some embodiments, the α-glucosidase inhibitor is acarbose, miglitol, L-arabinose, Gymnema sylvestre extract, Momoordica charantin extract, Trrigonellafoenum gracecum extract, Pterocarpus marsupium extract, Murraya koenigii extract, Ocimum sanctum extract, Tinospora cordifolia extract, Szygium cumini extract, Zingiber officinale extract, Allium sativum extract, or a mixture thereof.

In some embodiments, the glucose transport inhibitor is apple extract, phloridzin, Gynmerna sylvestre extract, strawberry extract, green coffee bean extract, or a mixture thereof.

In some embodiments, the fructose transport inhibitor is eucalyptus extract and coffee extract.

In some embodiments, the present disclosure provides a food or beverage additive comprising chromium and a carbohydrate blocker, wherein the carbohydrate blocker is an α-amylase inhibitor and an α-glucosidase inhibitor.

In some embodiments, the present disclosure provides a food or beverage additive comprising chromium and a carbohydrate blocker, wherein the carbohydrate blocker is selected from white kidney bean extract, L-arabinose, Hibiscus extract, Momordica charantia extract, Gymnema extract, apple extract, and eucalyptus extract.

In some embodiments, the carbohydrate blocker is white kidney bean extract. In some embodiments, the white kidney bean extract is extracted from Phaseolus vulgaris fruit. In some embodiments, the white kidney bean extract is a water extract from Phaseolus vulgaris fruit.

In some embodiments, the carbohydrate blocker is L-arabinose.

In some embodiments, the carbohydrate blocker is Hibiscus extract. In some embodiments, the Hibiscus extract is extracted from Hibiscus sabdariffa flower. In some embodiments, the Hibiscus extract is a water extract from Hibiscus sabdariffa flower.

in some embodiments, the carbohydrate blocker is white kidney bean extract and L-arabinose. In some embodiments, the carbohydrate blocker is Hibiscus extract and L-arabinose. In some embodiments, the carbohydrate blocker is white kidney bean extract, L-arabinose, and Hibiscus extract.

In some embodiments, the carbohydrate blocker is Momordica charantia extract and white kidney bean extract. In some embodiments, the carbohydrate blocker is Momordica charantia extract and L-arabinose. In some embodiments, the carbohydrate blocker is Momordica charantia extract and Hibiscus extract.

In some embodiments, the carbohydrate blocker is Momordica charantia extract, white kidney bean extract, and L-arabinose. In some embodiments, the carbohydrate blocker is Momordica charantia extract, white kidney bean extract, and Hibiscus extract. In some embodiments, the carbohydrate blocker is Momordica charantia extract, L-arabinose, and Hibiscus extract. In some embodiments, the carbohydrate blocker is Momordica charantia extract, L-arabinose, white kidney bean extract, and Hibiscus extract.

Pharmaceutical compositions comprising chromium, a carbohydrate blocker, and a pharmaceutically acceptable excipient may be useful for at least partially blocking digestion and adsorption of dietary carbohydrates.

In one aspect, the present disclosure provides a method for at least partially blocking digestion and adsorption of dietary carbohydrates comprising administering, to a subject in need thereof, a pharmaceutical composition described herein.

In some embodiments, at least about 0.5%, at least about 1%, at least about 2.5%, at least about 5%, at least about 10%, at least about 20%, at least about 30%, at least about 40%, or at least about 50% of dietary carbohydrates consumed within about 30 minutes after administration of the pharmaceutical composition are not absorbed.

In another aspect, the present disclosure provides a method of losing or maintaining body weight.

In another aspect, the present disclosure provides a method for supporting healthy blood sugar levels comprising administering, to a subject in need thereof, a pharmaceutical composition disclosed herein.

In some embodiments, said method results in lower blood sugar levels. In some embodiments, said method results in more consistent blood sugar levels. In some embodiments, said method reduces the severity or frequency of blood sugar spikes.

In another aspect, the present disclosure provides a method for treating, reducing the severity of, reducing the incidence of, delaying the onset of, or reducing pathogenesis of a chronic condition associated with metabolic syndrome comprising administering, to a subject in need thereof, a pharmaceutical composition described herein.

In some embodiments, the chronic condition associated with metabolic syndrome is selected from insulin resistance, type 2 diabetes, obesity, nonalcoholic fatty liver disease, chronic kidney disease, elevated blood pressure, polycystic ovary syndrome, cardiovascular disorders such as hypertension, and dyslipidemias such as high triglyceride and low HDL-cholesterol levels, acanthosis nigricans, hirsutism, peripheral neuropathy, and retinopathy.

In some embodiments, the chronic condition associated with metabolic syndrome is type 2 diabetes.

In some embodiments, the chronic condition associated with metabolic syndrome is prediabetes.

In some embodiments, the chronic condition associated with metabolic syndrome is high blood pressure.

In another aspect, the present disclosure provides a method for treating, reducing the severity of, reducing the incidence of, delaying the onset of, or reducing pathogenesis of insulin resistance comprising administering, to a subject in need thereof, a pharmaceutical composition of any one of claims 1 to 32.

In some embodiments, the method further comprising slowing the rate of aging in the subject in need thereof.

In some embodiments of all aspects, the daily dosage of chromium ranges from about 100 mcg to about 900 mcg. In some embodiments, the daily dosage of chromium is about 100 mcg, about 150 mcg, about 200 meg, about 250 mcg, about 300 meg, about 350 mcg, about 400 mcg, about 450 meg, about 500 mcg, about 550 mcg, about 600 meg, about 650 mcg, about 700 mcg, about 750 mcg, about 800 mcg, about 850 mcg, or about 900 mcg. In some embodiments, the daily dosage of chromium is about 500 mcg. In some embodiments, the daily dosage of chromium about 750 mcg.

In some embodiments of all aspects, the pharmaceutical composition comprises chromium, white kidney bean extract, L-arabinose, Hibiscus extract, and optionally Seville orange extract.

In some embodiments of all aspects, the daily dosage of white kidney bean extract ranges from about 600 mg to about 3000 mg. In some embodiments, the daily dosage of white kidney bean extract is about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg. In some embodiments, the daily dosage of white kidney bean extract is about 1000 mg. In some embodiments, the daily dosage of white kidney bean extract is about 1500 mg.

In some embodiments of all aspects, the daily dosage of L-arabinose is about 500 mg to about 1500 mg. In some embodiments, the daily dosage of L-arabinose is about 500 mg, about 600 mg, about 700 mg, about 800 mg, about 900 mg, about 1000 mg, about 1100 mg, about 1200 mg, about 1300 mg, about 1400 mg, or about 1500 mg. In some embodiments, the daily dosage of L-arabinose is about 1000 mg. In some embodiments, the daily dosage of L-arabinose is about 1500 mg.

In some embodiments of all aspects, the daily dosage of Hibiscus extract is about 200 mg to about 1500 mg. In some embodiments, the daily dosage of Hibiscus extract is about 200 mg, about 300 mg, about 400 mg, about 500 mg, about 600 mg, about 700 mg, about 800 mg, about 900 mg, about 1000 mg, about 1110 mg, about 1200 mg, about 1300 mg, about 1400 mg, or about 1500 mg. In some embodiments, the daily dosage of Hibiscus extract is about 400 mg. In some embodiments, the daily dosage of Hibiscus extract is about 600 mg.

In some embodiments of all aspects, the daily dosage of Momordica charantia extract is about 100 mg to about 4000 mg. In some embodiments, the daily dosage of Mornordica charantia extract is about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, or about 4000 mg. In some embodiments, the daily dosage of Momordica charantia extract is about 400 mg.

In some embodiments of all aspects, the daily dosage of Seville orange extract is about 200 mg to about 600 mg. In some embodiments, the daily dosage of Seville orange extract is about 200 mg, about 300 mg, about 400 mg, about 500 mg, or about 600 mg. In some embodiments, the daily dosage of Seville orange extract is about 400 mg. In some embodiments, the daily dosage of Seville orange extract is about 600 mg.

In some embodiments of all aspects, the pharmaceutical composition further comprises one or more of protein, lipids, and carbohydrates.

In some embodiments of all aspects, the pharmaceutical composition is administered in combination with a meal.

In some embodiments of all aspects, the pharmaceutical composition is administered in combination with a meal high in calories and/or carbohydrates.

in some embodiments of all aspects, the pharmaceutical composition is administered two to three times daily.

In some embodiments of all aspects, the pharmaceutical composition is administered two to three times daily with a meal. In some embodiments of all aspects, the pharmaceutical composition is administered two times daily with a meal.

In some embodiments of all aspects, the pharmaceutical composition is administered two times daily with a meal, wherein the two meals are those most likely to be high in carbohydrates.

In some embodiments of all aspects, the pharmaceutical composition is administered two times daily with a meal, wherein the two meals are those most likely to be high in calories.

In some embodiments of all aspects, the pharmaceutical composition is administered two times daily with a meal, wherein the two meals are those most likely to be high in carbohydrates and/or calories.

In some embodiments of all aspects, the pharmaceutical composition is administered two times daily, once with breakfast and once with lunch. In some embodiments of all aspects, the pharmaceutical composition is administered two times daily with a meal, once with lunch and once with dinner. In some embodiments of all aspects, the pharmaceutical composition is administered three times daily with a meal.

In some embodiments of all aspects, the subject in need thereof has pre-diabetic fasting blood glucose levels. In some other embodiments, the subject in need thereof has diabetic fasting blood glucose levels.

In some embodiments of all aspects, the subject in need thereof has insulin resistance.

In some embodiments of all aspects, the subject in need thereof has metabolic syndrome.

In some embodiments of all aspects, the subject in need thereof has type 2 diabetes or prediabetes.

A person of skill in the art will appreciate that concentration active ingredients in an extract vary and that modifications to the daily dosage ranges for extracts may be required to account for batch-to-batch and source-to-source variability in extract concentrations.

### EXAMPLES

### Example 1: Administration

A subject with pre-diabetic fasting glucose levels may lower his or her blood sugar level and support insulin function and activity by taking three capsules two times daily with a meal, wherein the three capsules (one dosage) comprise:

250 mcg chromium polynicotinate;

500 mg white kidney bean extract from Phaseolus vulgaris fruit;

500 mg L-arabinose;

200 mg Hibiscus extract from Hibiscus sabdariffa flower; and

200 mg Seville orange extract from Citrus aurantium fruit; or

the three capsules (one dosage) comprise:

100 mcg chromium polynicotinate;

500 mg white kidney bean extract from Phaseolus vulgaris fruit;

500 mg L-arabinose;

200 mg Hibiscus extract from Hibiscus sabdarffa flower;

200 mg Momordica charantia extract; and

200 mg Seville orange extract from Citrus aurantium fruit

In some instances, the subject may take the three capsules with the two meals in his or her day most likely to be high in calories and carbohydrates. The two meals may be selected based on personal preference, local culture, and/or personal dietary habits. As a non-limiting example, a subject in the United States may take the three capsules twice daily with lunch and dinner. In another non-limiting example, a subject in the Mexico may take the three capsules twice daily with breakfast and lunch.

### Example 2: Capsule Manufacturing

A capsule comprising chromium polynicotinate, white kidney bean extract from Phaseolus vulgaris fruit, L-arabinose, Hibiscus extract from Hibiscus sabdariffa flower, Seville orange extract from Citrus aurantium fruit, and optionally Momordica charantia extract may be prepared by obtaining chromium polynicotinate, white kidney bean extract, L-arabinose, Hibiscus extract, Seville orange extract, and optionally Momordica charantia extract in powder form from commercial suppliers. The active ingredients may be analyzed to evaluate quality. Non-limiting analysis examples include physical characteristics analysis (e.g., organoleptic analyses and particle size analysis), hygienic analysis (e.g., total plate count for microorganisms), and purity and potency analysis (e.g., HPLC, atomic absorption, and other analytical techniques). After analysis, the active ingredients are weighed, double-checked for proper amounts, and blended with pharmaceutically acceptable excipients until the powder mix is homogeneous. Non-limiting examples of pharmaceutically acceptable excipients include microcrystalline cellulose, magnesium stearate, and silicon dioxide. The powder is transferred to encapsulating machinery, and gelatin capsules are filled with said powder, dusted, polished, and check-weighed. Sample capsules are randomly selected and analyzed for specific nutrients or related ingredients, as well as for meeting quality and safety standards.

### Other Embodiments

While the foregoing disclosure has been described in some detail for purposes of clarity and understanding, the disclosure is to be considered as illustrative and not restrictive. The skilled artisan reading this disclosure will appreciate that various changes in form and detail can he made without departing from the scope of the disclosure. Thus, the scope of the disclosure should be determined with reference to the following appended claims and embodiments, along with the full scope of equivalents to which the claims are entitled.

Embodiment 1. A pharmaceutical composition comprising chromium, a carbohydrate blocker, and a pharmaceutically acceptable excipient.

Embodiment 2. The pharmaceutical composition of embodiment 1, wherein the chromium is chromium nicotinate, chromium polynicotinate, chromium picolinate, chromium histidinate, or a mixture thereof.

Embodiment 3. The pharmaceutical composition of embodiment 1 or 2, wherein the chromium is chromium polynicotinate.

Embodiment 4. The pharmaceutical composition of any one of embodiments 1 to 3, wherein the carbohydrate blocker is an α-amylase inhibitor, an α-glucosidase inhibitor, a glucose transport inhibitor, a fructose transport inhibitor, or a mixture thereof.

Embodiment 5. The pharmaceutical composition of any one of embodiments 1 to 4, wherein the carbohydrate blocker is an α-amylase inhibitor and an α-glucosidase inhibitor.

Embodiment 6. The pharmaceutical composition of any one of embodiments 1 to 4, wherein the carbohydrate blocker is selected from white kidney bean extract, L-arabinose, Hibiscus extract, Momordica charantia extract, Gymnema extract, apple extract, and eucalyptus extract.

Embodiment 7. The pharmaceutical composition of any one of embodiments 1 to 4 or 6, wherein the carbohydrate blocker is white kidney bean extract.

Embodiment 8. The pharmaceutical composition of any one of embodiments 1 to 4 or 6, wherein the carbohydrate blocker is L-arabinose.

Embodiment 9. The pharmaceutical composition of any one of embodiments 1 to 4 or 6, wherein the carbohydrate blocker is Hibiscus extract.

Embodiment 10. The pharmaceutical composition of any one of embodiments 1 to 4 or 6, wherein the carbohydrate blocker is Momordica charantia extract.

Embodiment 11. The pharmaceutical composition of any one of embodiments 1 to 6, wherein the carbohydrate blocker is white kidney bean extract and L-arabinose.

Embodiment 12. The pharmaceutical composition of any one of embodiments 1 to 6, wherein the carbohydrate blocker is Hibiscus extract and L-arabinose.

Embodiment 13. The pharmaceutical composition of any one of embodiments 1 to 6, wherein the carbohydrate blocker is white kidney bean extract, L-arabinose, and Hibiscus extract.

Embodiment 14. The pharmaceutical composition of any one of embodiments 1 to 6, wherein the carbohydrate blocker is white kidney bean extract, L-arabinose, Momordica charantia extract, and Hibiscus extract.

Embodiment 15. The pharmaceutical composition of any one of embodiments 1 to 14, wherein the chromium is present in an amount ranging from about 50 mcg to about 300 mcg.

Embodiment 16. The pharmaceutical composition of any one of embodiments 1 to 15, wherein the chromium is present in an amount of about 250 mcg.

Embodiment 17. The pharmaceutical composition of any one of embodiments 6, 11, 13, or 14, wherein the white kidney bean extract is present in an amount ranging from about 300 mg to about 1000 mg.

Embodiment 18. The pharmaceutical composition of embodiment 17, wherein the white kidney bean extract is present in an amount of about 500 mg.

Embodiment 19. The pharmaceutical composition of any one of embodiment 8 or 11-14, wherein the L-arabinose is present in an amount ranging from about 250 mg to about 500 mg.

Embodiment 20. The pharmaceutical composition of embodiment 19, wherein the L-arabinose is present in an amount of about 500 mg.

Embodiment 21. The pharmaceutical composition of any one of embodiments 9 or 12-14, wherein the Hibiscus extract is present in an amount ranging from about 100 mg to about 500 mg.

Embodiment 22. The pharmaceutical composition of embodiment 21, wherein the Hibiscus extract is present in an amount of about 200 mg.

Embodiment 23. The pharmaceutical composition of embodiment 10 or 14, wherein the Momordica charantia extract is present in an amount ranging from about 50 mg to about 2000 mg.

Embodiment 24. The pharmaceutical composition of embodiment 23, wherein the Momordica charantia extract is present in an amount of about 200 mg.

Embodiment 25. A pharmaceutical composition of embodiment 1 comprising:

about 75 mcg to about 300 mcg chromium;

about 300 mg to about 1000 mg white kidney bean extract;

about 250 mg to about 500 mg L-arabinose;

about 100 mg to about 500 mg Hibiscus extract; and

a pharmaceutically acceptable excipient.

Embodiment 26. The pharmaceutical composition of embodiment 1, further comprising about 50 mg to about 2000 mg Momordica charantia extract.

Embodiment 27. A pharmaceutical composition of embodiment 25 comprising:

about 250 mcg chromium;

about 500 mg white kidney bean extract;

about 500 mg L-arabinose;

about 200 mg Hibiscus extract; and

a pharmaceutically acceptable excipient.

Embodiment 28. A pharmaceutical composition of embodiment 26 comprising:

about 100 mcg chromium;

about 500 mg white kidney bean extract;

about 500 mg L-arabinose;

about 200 mg Hibiscus extract;

about 200 mg Momordica charantia extract; and

a pharmaceutically acceptable excipient.

Embodiment 29. The pharmaceutical composition of any one of embodiments 25 to 28, wherein the chromium is chromium polynicotinate.

Embodiment 30. The pharmaceutical composition of any one of embodiments 1 to 29, further comprising an energy-boosting additive.

Embodiment 31. The pharmaceutical composition of embodiment 30, wherein the energy-boosting additive is caffeine, guarana, green tea extract, ginseng, coffee extract, black tea extract, Oolong tea extract, vitamin B12, coenzyme Q12, Seville orange extract, or a mixture thereof.

Embodiment 32. The pharmaceutical composition of embodiment 31, wherein the energy-boosting additive is Seville orange extract.

Embodiment 33. The pharmaceutical composition of embodiment 32, wherein the Seville orange extract is present in an amount ranging from about 100 mg to about 200 mg.

Embodiment 34. The pharmaceutical composition of embodiment 33, wherein the Seville orange extract is present in an amount of about 200 mg.

Embodiment 35. The pharmaceutical composition of any one of embodiments 1 to 34, wherein the pharmaceutical composition is formulated as a solid oral dosage form.

Embodiment 36. The pharmaceutical composition of any one of embodiments 1 to 35, wherein the pharmaceutical composition is formulated as three solid oral dosage forms.

Embodiment 37. The pharmaceutical composition of any one of embodiments 1 to 36, wherein the solid oral dosage form is a capsule.

Embodiment 38. The pharmaceutical composition of any one of embodiments 1 to 34, wherein the pharmaceutical composition is formulated as a powder.

Embodiment 39. The food or beverage additive comprising chromium and a carbohydrate blocker.

Embodiment 40. The food or beverage additive of embodiment 39, wherein the chromium is chromium nicotinate, chromium polynicotinate, chromium picolinate, chromium histidinate, or a mixture thereof.

Embodiment 41. The food or beverage additive of embodiment 39 or 40, wherein the chromium is chromium polynicotinate.

Embodiment 42. The food or beverage additive of any one of embodiments 39 to 41, wherein the carbohydrate blocker is an α-amylase inhibitor, an α-glucosidase inhibitor, a glucose transport inhibitor, a fructose transport inhibitor, or a mixture thereof.

Embodiment 43. The food or beverage additive of any one of embodiments 39 to 42, wherein the carbohydrate blocker is an α-amylase inhibitor and an α-glucosidase inhibitor.

Embodiment 44. The food or beverage additive of any one of embodiments 39 to 43, wherein the carbohydrate blocker is selected from white kidney bean extract, L-arabinose, Hibiscus extract, Momordica charantia extract, Gymnema extract, apple extract, and eucalyptus extract.

Embodiment 45. The food or beverage additive of embodiment 44, wherein the carbohydrate blocker is white kidney bean extract.

Embodiment 46. The food or beverage additive of embodiment 44, wherein the carbohydrate blocker is L-arabinose.

Embodiment 47. The food or beverage additive of embodiment 44, wherein the carbohydrate blocker is Hibiscus extract.

Embodiment 48. The food or beverage additive of embodiment 44, wherein the carbohydrate blocker is Momordica charantia extract.

Embodiment 49. The food or beverage additive of embodiment 44, wherein the carbohydrate blocker is white kidney bean extract and L-arabinose.

Embodiment 50. The food or beverage additive of embodiment 44, wherein the carbohydrate blocker is Hibiscus extract and L-arabinose.

Embodiment 51. The food or beverage additive of embodiment 44, wherein the carbohydrate blocker is white kidney bean extract, L-arabinose, and Hibiscus extract.

Embodiment 52. The food or beverage additive of embodiment 44, wherein the carbohydrate blocker is white kidney bean extract, L-arabinose, Momordica charantia extract, and Hibiscus extract.

Embodiment 53. A method for supporting healthy blood sugar levels comprising administering, to a subject in need thereof, a pharmaceutical composition of any one of embodiments 1 to 38.

Embodiment 54. A method for at least partially blocking digestion and adsorption of dietary carbohydrates comprising administering, to a subject in need thereof, a pharmaceutical composition of any one of embodiments 1 to 38.

Embodiment 55. A method for treating, reducing the severity of, reducing the incidence of, delaying the onset of, or reducing pathogenesis of a chronic condition associated with metabolic syndrome comprising administering, to a subject in need thereof, a pharmaceutical composition of any one of embodiments 1 to 38.

Embodiment 56. The method of embodiment 55, wherein the chronic condition associated with metabolic syndrome is type 2 diabetes.

Embodiment 57. The method of embodiment 55, wherein the chronic condition associated with metabolic syndrome is high blood pressure.

Embodiment 58. A method for treating, reducing the severity of, reducing the incidence of, delaying the onset of, or reducing pathogenesis of insulin resistance comprising administering, to a subject in need thereof, a pharmaceutical composition of any one of embodiments 1 to 38.

Embodiment 59. The method of embodiment 58, further comprising slowing the rate of aging in the subject in need thereof.

Embodiment 60. The method of any one of embodiments 53 to 59, wherein the subject in need thereof exhibits diabetic fasting glucose levels.

Embodiment 61. The method of any one of embodiments 53-55 or 57-59, wherein the subject in need thereof exhibits non-diabetic fasting glucose levels.

Embodiment 62. The method of any one of embodiments 53 to 61, wherein the HDL levels for the subject also improve as a result of treatment.

Embodiment 63. The method of any one of embodiments 53 to 62, wherein the daily dosage of chromium ranges from about 100 mcg to about 900 meg.

Embodiment 64. The method of any one of embodiments 45 to 54, wherein the pharmaceutical composition comprises chromium, white kidney bean extract, L-arabinose, Hibiscus extract, and optionally Momordica charantia extract and/or Seville orange extract.

Embodiment 65. The method of embodiment 64, wherein the daily dosage of white kidney bean extract ranges from about 600 mg to about 3000 mg.66. The method of embodiment 64, wherein the daily dosage of L-arabinose is about 500 mg to about 1500 mg.

Embodiment 67. The method of embodiment 64, wherein the daily dosage of Hibiscus extract is about 200 mg to about 1500 mg.

Embodiment 68. The method of embodiment 64, wherein the daily dosage of Seville orange extract is about 200 mg to about 600 mg.

Embodiment 69. The method of embodiment 64, wherein the daily dosage of Momordica charantia extract is about 100 mg to about 4000 mg.

Embodiment 70. The method of any one of embodiments 53 to 69, wherein the pharmaceutical composition is administered in combination with a meal.

Embodiment 71. The method of any one of embodiments 53 to 69, wherein the pharmaceutical composition is administered two to three times daily.

Embodiment 72. The method of any one of embodiments 53 to 69, wherein the pharmaceutical composition is administered two to three times daily with a meal.

1-72. (canceled)

73. A method for treating fatty liver disease in a subject in need thereof, the method comprising administering to the subject a pharmaceutical composition comprising chromium, white kidney bean extract, L-arabinose, Hibiscus extract, and a pharmaceutically acceptable excipient.

74. The method according to claim 73, wherein the chromium comprises chromium nicotinate, chromium polynicotinate, chromium picolinate, chromium histidinate, or a mixture thereof.

75. The method according to claim 73, wherein the chromium is present in the pharmaceutical composition in an amount of about 50 mcg to about 300 mcg.

76. The method according to claim 75, wherein the chromium is present in the pharmaceutical composition in an amount of about 250 mcg.

77. The method according to claim 73, wherein the white kidney bean extract is present in the pharmaceutical composition in an amount of about 300 mg to about 1000 mg.

78. The method according to claim 77, wherein the white kidney bean extract is present in the pharmaceutical composition in an amount of about 500 mg.

79. The method according to claim 73, wherein the L-arabinose is present in the pharmaceutical composition in an amount of about 250 mg to about 500 mg.

80. The method according to claim 79, wherein the L-arabinose is present in the pharmaceutical composition in an amount of about 500 mg.

81. The method according to claim 73, wherein the Hibiscus extract is present in the pharmaceutical composition in an amount of about 100 mg to about 500 mg.

82. The method according to claim 81, wherein the Hibiscus extract is present in the pharmaceutical composition in an amount of about 200 mg.

83. The method according to claim 73, wherein the pharmaceutical composition comprises:
about 75 mcg to about 300 mcg chromium;
about 300 mg to about 1000 mg white kidney bean extract;
about 250 mg to about 500 mg L-arabinose;
about 100 mg to about 500 mg Hibiscus extract; and
a pharmaceutically acceptable excipient.

84. The method according to claim 73, wherein the pharmaceutical composition comprises:
about 250 mcg chromium;
about 500 mg white kidney bean extract;
about 500 mg L-arabinose;
about 200 mg Hibiscus extract; and
a pharmaceutically acceptable excipient.

85. The method according to claim 73, wherein the pharmaceutical composition further comprises Momordica charantia extract, Gymnema extract, apple extract, and/or eucalyptus extract.

86. The method according to claim 85, wherein the pharmaceutical composition further comprises Momordica charantia extract and the Momordica charantia extract is present in the pharmaceutical composition in an amount of about 50 mg to about 2000 mg.

87. The method according to claim 86, wherein the Momordica charantia extract is present in the pharmaceutical composition in an amount of about 200 mg.

88. The method according to claim 87, wherein the pharmaceutical composition comprises:
about 100 mcg chromium;
about 500 mg white kidney bean extract;
about 500 mg L-arabinose;
about 200 mg Hibiscus extract;
about 200 mg Momordica charantia extract; and
a pharmaceutically acceptable excipient.

89. The method according to claim 73, wherein the pharmaceutical composition further comprises an energy-boosting additive.

90. The method according to claim 89, wherein the energy-boosting additive comprises caffeine, guarana, green tea extract, ginseng, coffee extract, black tea extract, Oolong tea extract, vitamin B12, coenzyme Q12, Seville orange extract, or a mixture thereof.

91. The method according to claim 90, wherein the energy-boosting additive comprises Seville orange extract and the Seville orange extract is present in the pharmaceutical composition in an amount of about 100 mg to about 200 mg.

92. The method according to claim 91, wherein the Seville orange extract is present in the pharmaceutical composition in an amount of about 200 mg.

93. The method according to claim 73, wherein the pharmaceutical composition is formulated as a solid oral dosage form.

94. The method according to claim 73, wherein the pharmaceutical composition is formulated as a powder.

95. The method according to claim 93, wherein the solid oral dosage form is encapsulated in a capsule.

96. The method according to claim 93, wherein the solid oral dosage is encapsulated in at least three capsules.

97. The method according to claim 73, comprising administering the pharmaceutical composition in combination with a meal.

98. The method according to claim 73, comprising administering the pharmaceutical composition two to three times daily.

99. The method according to claim 73, comprising administering the pharmaceutical composition in combination with a meal two to three times daily.
